(19) United States
(12) Patent Application Publication
(10) Pub. No.: US 2017/0258718 A1
(43) Pub. Date: Sep. 14, 2017

(54) PHARMACEUTICAL COMPOSITION, PREPARATION AND USES THEREOF

(71) Applicant: NANOBIOTIX, PARIS (FR)

(72) Inventors: MARIE-EDITH MEYRE, SAINT MANDE (FR); AGNES POTTIER, PARIS (FR); LAURENT LEVY, PARIS (FR)

(21) Appl. No.: 15/529,102

(22) PCT Filed: Nov. 24, 2015
(86) PCT No.: PCT/EP2015/077438

§ 371 Date: May 24, 2017

(30) Foreign Application Priority Data
Nov. 25, 2014 (EP) 14306873.2

(51) Int. Cl. A61K 9/127; A61K 31/704; A61K 9/51

(52) U.S. Cl. CPC A61K 9/127; A61K 9/51; A61K 31/704

(57) ABSTRACT

The present invention relates to a pharmaceutical composition comprising the combination of (i) at least one biocompatible nanoparticle and (ii) at least one pharmaceutical compound, to be administered to a subject in need of such a pharmaceutical compound, wherein the nanoparticle potentiates the pharmaceutical compound efficiency. The longest dimension of the biocompatible nanoparticle is typically between about 4 and about 500 nm, its absolute surface charge value is of at least 10 mV (|10 mV|), and its Young modulus is less than 100 kPa. The invention also relates to such a composition for use for administering the pharmaceutical compound in a subject in need thereof, wherein the at least one nanoparticle and the at least one pharmaceutical compound are to be administered in the subject between more than 5 minutes and about 72 hours one from each other.

### FIELD OF THE INVENTION

The invention relates to a pharmaceutical composition comprising the combination of (i) at least one biocompatible nanoparticle and of (ii) at least one compound of interest, typically at least one pharmaceutical compound, to be administered to a subject in need of such a compound of interest, wherein the nanoparticle potentiates the compound(s) of interest efficiency. The longest dimension of the at least one biocompatible nanoparticle is typically between about 4 and about 500 nm, its absolute surface charge value is of at least 10 mV (|10 mV|), and its Young modulus is less than 100 kPa.

The invention also relates to such a composition for use for administering the at least one compound of interest in a subject in need thereof, wherein the at least one biocompatible nanoparticle and the at least one compound of interest are to be administered in said subject sequentially, typically between more than 5 minutes and about 72 hours one from each other.

The combined, and typically sequential, administration to the subject of the at least one biocompatible nanoparticle and of the at least one compound of interest maintains the pharmaceutical (i.e. therapeutic, prophylactic or diagnostic) benefit of said compound of interest for a reduced toxicity thereof in said subject, or increases its pharmaceutical benefit for an equivalent or reduced toxicity, when compared to the pharmaceutical benefit and toxicity induced by said compound when administered at the standard pharmaceutical dose. The pharmaceutical composition of the invention typically allows a reduction of at least 10% of the administered compound of interest's pharmaceutical dose when compared to the standard pharmaceutical dose of said compound while maintaining the same pharmaceutical benefit for an equivalent toxicity, preferably a reduced toxicity, for the subject, or while increasing the pharmaceutical benefit for an equivalent or reduced toxicity for the subject.

### BACKGROUND

In order to ensure safety and efficacy, pharmaceutic compounds are required to be selectively delivered to their target site at an optimal rate in the subject in need thereof Pharmacokinetics (pK) is a branch of pharmacology dedicated to the determination of the fate of substances administered externally to a living organism. This determination involves steps of measuring compound's concentrations in all major tissues over a long enough period of time, preferably until the compound's elimination. Pharmacokinetics is necessary to efficiently describe the compound's behavior in vivo, including the mechanisms of its absorption and distribution as well as its chemical changes in the organism. The pK profile in the blood can be fitted using various programs to obtain key pK parameters that quantitatively describe how the body handles the compound. Important parameters include maximum concentration (Cₘₐₓ), half-life (t_{1/2}), clearance, area under curve (AUC), and mean resident time (MRT), i.e. the average time during which a compound stays in an organism. When a prolonged blood circulation of the compound formulation is observed, it is usually associated to an increased t_{1/2}, a reduced clearance, an increased AUC, and an increased MRT. pK data are often used in deciding the optimal dose and dose regimen for maintaining the desirable blood concentration in order to improve therapeutics' efficiency with minimal side effects. In addition, as well known by the skilled person, the blood concentration of a compound is correlated with both its efficacy and toxicity in most cases, typically for free drugs.

The physico-chemical properties of therapeutic as well as prophylactic compounds have an important impact on their pharmacokinetic and metabolic fate in the body. Therefore, selection of appropriate physico-chemical properties is key when designing such a compound. However, since the compound is not always endogenously provided by the organism itself and is usually externally administered, its biodistribution profile has to be optimized in order to fit with, and preferably optimize, the desired pharmacological action thereof.

Several approaches have been explored to optimize the delivery of a compound to its target site. A strategy is to design a therapeutic compound with stealth properties to prolong its blood half-life and consequently, to enhance its accumulation to the target site. One favorable approach is the covalent attachment of polyethylene glycol (PEG) to the therapeutic compound that has proved to increase the in vivo half-life (t_{1/2}) of the circulating compound, the level of the in vivo half-life increase varying depending partly on the nature of the compound and on that of the coating. Also, drug carriers such as liposomes, emulsions or micelles have been developed to enhance therapeutic efficacy of drugs by modifying their biodistribution profile in the subject's body. However, lack of selectivity in the biodistribution of the therapeutic compounds still remains a concern. So far, poor pharmacokinetics and high toxicity are important causes of failure in therapeutic compounds development.

As an example, in the context of cancer treatment, intentional inhibition of essential functions of the body in order to kill cancer cells results in toxicity in normal cells, and clinicians have to rely on differences in dose-response and therapeutic compounds distribution between tumors and normal tissues to find a possible therapeutic window. Of note, hepatotoxicity remains a major reason for drug withdrawal from pharmaceutical development and clinical use due to direct and indirect mechanisms of drug-induced cell injury in the liver.

An approach proposed for nanoparticulate compounds such as drug carriers [Critical Reviews in Therapeutic Drug Carrier Systems 11(1):31-59 1994] is to pre-inject a decoy carrier to decrease, saturate, or even inactivate the phagocytic capacity of the reticuloendothelial system (RES). Impairment or blockade may also be associated with decreased plasma levels of opsonic molecules. Intravenous administration of certain agents, such as alkyl esters of fatty acids, dextran sulfate, salts of rare earth elements (e.g. GdCl₃), drug carriers, either empty or encapsulating clodronate, prior to administration of test particles, has been demonstrated to induce moderate to dramatic reduction in kupffer cells uptake.

For instance, authors in “Biomimetic amplification of nanoparticle homing to tumors” [PNAS 2007], reported the role of RES in the clearance of their nanoparticles “CREKA-SPIO”. Initial experiments showed that intravenous (IV) injected “CREKA-SPIO” nanoparticles did not effectively accumulate in MDA-MB-435 breast cancer xenografts. In contrast, a high concentration of particles was seen in RES tissues. By depleting RES macrophages in the liver with liposomal clodronate, they found a 5-fold prolongation of their particle's half-life. However, clodronate agent induces the apoptosis of macrophages from liver and spleen, and this is considered as globally detrimental as macrophages depletion increases the risks associated to immunosupression and infection. In a second experiment, the authors tested liposomes coated with chelated Ni (II) as a potential decoy particle hypothesizing that iron oxide and Ni (II) would attract similar plasma opsonins, and that Ni-liposomes could therefore deplete them in the systemic circulation. Indeed, intravenous (IV) injected Ni-liposomes, whether administered 5 minutes or 48 hours before the injection of CREKA-SPIO nanoparticles, allows a five-fold increase of the nanoparticles' blood half-life. However, high toxicity was observed causing deaths among tumor mice. Plain liposomes were also tested instead of Ni-liposomes. However, while reducing the toxicity when compared to said Ni-liposomes, plain liposomes were far less effective than them. Indeed, the blood half-life increase was only of a factor about 2.

WO2005086639 relates to methods of administering a desired agent selectively to a target site in a subject, typically in the context of ultrasound or X-ray exposure, or in the context of magnetic resonance imaging (MRI), as well as in the context of therapy. The aim of the described method is to improve or maintain the efficiency of the agent of interest while reducing the total dose of agents concretely administered thanks to concomitant administration of a decoy inactive carrier.

The described invention employs a probability-based approach. A non-targeted inactive agent (“inactive carrier”) is co-administered (i.e. “substantially simultaneously”) with a targeted agent of interest (present in an “active composition”) exhibiting similar physical features, in order to facilitate the evasion of the RES system by the targeted agent of interest thereby allowing an improved uptake of the agent of interest at the desired site. This approach results in a lower exposure of patients to the agent of interest and, as a consequence, in a lower per dosage cost of said agent of interest. The active composition and the decoy inactive carrier are administered within five minutes of each other, preferably within 2 minutes of each other, or even less. This approach relies on the presence of a large excess of untargeted “carrier” or “decoy” vehicles and on the probability that this decoy carrier in excess will compete with the targeted agent of interest for an uptake by the reticuloendothelial system when supplied in the presence of vehicles that are targeted to a desired location. The half-life of particles captured by RES is dose dependent, i.e. the circulating half-life of particles increases as the dosage increases. The slower clearance associated to higher dosages is thought to favor the maintaining of a total agents high concentration allowing a decrease of the dose of the agent of interest which is to be administered. In other words, an increased half-life of total agents due to a global higher dosage thereof should be beneficial to the targeted agents, according to the authors of WO2005086639. The requirement involved by this approach is that the active agent and the inactive one behave similarly with regard to their clearance characteristics in the RES, whatever their respective compositions.

In this approach, the quasi-concomitant injection of the inactive agent and of the active one is required to increase the global amount of agents present in the blood and consequently to prolong their blood half-life. Such strategy, which expressly relies on a probability-based approach, necessarily requires the association of the active agent with a targeting agent in order to achieve its successful accumulation on the target site by conferring said active agent an advantage over the inactive one. In addition, due to the quasi-concomitant injection, a specific design of the inactive carrier may be required depending on the intended use of the active composition.

WO2005/063305 relates to an assembly comprising a gas-filled microvesicle (with a size typically of at least 0.5 μm) and a component (with a size about below 100 nm) associated to said microvesicle. The resulting assembly is to be used as a pharmaceutically active component in diagnostically and/or therapeutically active formulations. The two components, i.e. the gas-filled microvesicle and the microvesicle associated component, are administered simultaneously typically for enhancing the imaging in the field of ultrasound contrast imaging, including targeted ultrasound imaging, ultrasound-mediated drug delivery and other imaging techniques.

As apparent from the prior art and despite of a long medical need, the improvement of the efficiency of compounds (including therapeutic, prophylactic as well as diagnostic compounds) which cannot be optimally used in patients due to their unacceptable toxicity or to their unfavorable pharmacokinetics parameters remains a concern.

### DETAILED DESCRIPTION

The present invention now allows optimization of the efficiency of compound(s) of interest (herein also simply identified as “compound(s)”) whatever its intended use in the context of therapy, prophylaxis or diagnostic. The composition herein described which is a combination of (i) at least one biocompatible nanoparticle and of (ii) at least one compound of interest, typically at least one pharmaceutical compound, optimize the at least one compound of interest's pharmacokinetic parameters, and, as a consequence, now renders possible the development of pharmaceutic compounds which could not have been developed otherwise due for example to their unacceptable toxicity. Typically, the biocompatible nanoparticle is not used as such as a pharmaceutical compound, i.e. as a therapeutic, prophylactic or diagnostic compound.

A typical composition of the invention (herein generally identified as “pharmaceutical composition”) is a composition comprising the combination of (i) at least one biocompatible nanoparticle and (ii) of at least one compound (“the compound of interest”), wherein the longest or largest dimension of the biocompatible nanoparticle is typically between about 4 nm and about 500 nm, the absolute surface charge value of the biocompatible nanoparticle is of at least 10 mV, and the Young modulus of the biocompatible nanoparticles is less than 100 kPa.

Typically, the ratio between the (at least one) biocompatible nanoparticles and compounds of interest is between 0.1/1 and 1000/1 or 0.5/1 and 1000/1, preferably between 0.5/1 and 500/1, even more preferably between 0.5/1 and 300/1.

The terms “about” and “around” when associated to a value such as for example a nanoparticle' size or a time interval indicates that a variation with the indicated value, which would be recognized by the skilled person as small variation, does not substantially impact the properties of the subject-matter it is associated to and that said subject-matter remains in the spirit of the claimed invention.

A preferred object of the invention is a pharmaceutical composition comprising the combination of (i) at least one biocompatible nanoparticle and of (ii) at least one pharmaceutical compound, wherein the longest or largest dimension of the biocompatible nanoparticle is between about 4 nm and about 500 nm, the absolute surface charge value of the biocompatible nanoparticle is of at least 10 mV (|10 mV|), and the Young modulus of the biocompatible nanoparticle is less than 100 kPa, for use for administering the at least one pharmaceutical compound in a subject in need thereof, wherein the at least one nanoparticle and the at least one pharmaceutical compound are to be administered in a subject in need of said pharmaceutical compound, between more than 5 minutes and about 72 hours one from each other, and wherein the biocompatible nanoparticle is not used as such as a pharmaceutical compound.

The combined and sequential administration to the subject of the biocompatible nanoparticle(s) and of the pharmaceutical compound(s), through the composition of the invention, typically allows (maintains) the same pharmaceutical (i.e. therapeutic, prophylactic or diagnostic) benefit of the compound(s) for a reduced toxicity thereof for the subject, or increase the pharmaceutical benefit of the compound(s) for an equivalent or reduced toxicity thereof for the subject (preferably a reduced toxicity), when compared to pharmaceutical benefit and toxicity induced by the standard pharmaceutical dose of said compound(s), typically in the absence of any nanoparticle.

The pharmaceutical composition of the invention typically allows a reduction of at least 10%, preferably at least 15%, of the administered compound(s) pharmaceutical (i.e. therapeutic, prophylactic or diagnostic) dose(s) when compared to standard pharmaceutical dose(s) of said compound(s) (i) while maintaining the same pharmaceutical benefit for an equivalent toxicity, preferably a reduced toxicity, for the subject or (ii) while increasing the pharmaceutical benefit for an equivalent or reduced toxicity for the subject, typically in the absence of any nanoparticle.

As the shape of the particle can influence its “biocompatibility”, particles having a quite homogeneous shape are herein preferred. For pharmacokinetic reasons, nanoparticles being essentially spherical/round or ovoid in shape are thus preferred. Such a shape also favors the nanoparticle interaction with or uptake by cells. Spherical/round shape is particularly preferred.

In the spirit of the invention, the term “nanoparticle” refers to a product, in particular a synthetic product, with a size in the nanometer range, typically between about 1 nm and about 500 nm, preferably between about 4 nm and about 500 nm, between about 4 and about 400 nm, about 30 nm and about 300 nm, about 20 nm and about 300 nm, about 10 nm and about 300 nm, for example between about 4 nm and about 100 nm, for example between about 10 nm, 15 nm or 20 nm and about 100 nm, or between about 100 nm and about 500 nm, typically between about 100 nm and about 300 nm.

The terms “size of the nanoparticle”, “largest size of the nanoparticle” and “longest size of the nanoparticle” herein typically refer to the “longest or largest dimension of the nanoparticle” or “diameter of the nanoparticle” when spherical/round or ovoid in shape. Transmission Electron Microscopy (TEM) or Cryo-TEM can be used to measure the size of the nanoparticle. As well, Dynamic Light Scattering (DLS) can be used to measure the hydrodynamic diameter of nanoparticles in solution. These two methods may further be used one after each other to compare the hydrodynamic diameter of a nanoparticle measured by DLS with the size of said nanoparticle measured by TEM or Cryo-TEM, in order to confirm said size. A preferred method is DLS (Ref. International Standard ISO22412 Particle Size Analysis—Dynamic Light Scattering, International Organisation for Standardisation (ISO) 2008).

To be usable in the context of the invention, the absolute electrostatic surface charge (also herein identified as “charge” or “surface charge”) of the biocompatible nanoparticle is to be higher than 110 mV1 (absolute value). The surface charge of a nanoparticle is typically determined by zeta potential measurements in aqueous medium for a nanoparticles concentration between 0.2 and 10 g/L, for a pH between 6 and 8, and typically for electrolytes concentrations in the aqueous medium between 0.001 and 0.2 M, for example 0.01 M or 0.15 M.

Typically, the biocompatible nanoparticle of the present invention has an electrostatic surface charge of at least |10 mV|, i.e. below −10 mV or above +10 mV, for example below between −12 mV or −15 mV and −20 mV or above between +12 mV or +15 mV and +20 mV, typically below −15 mV or above +15 mV. Preferably, the biocompatible nanoparticle of the present invention has an absolute electronic surface charge value (“absolute surface charge value”) of more than 10 mV, said charge being even more preferably a negative charge.

To be usable in the context of the present invention, the Young modulus of the biocompatible nanoparticle, which reflects the elasticity of the biocompatible nanoparticle, is to be less than 500 kPa, typically less than 400 kPa, more preferably less than 300 or 200 kPa, preferably less than100 kPa. The Young modulus of the bio compatible nanoparticle can for example be less than 70 kPa, or less than 60, 50, 40, 30, 20 or 10 kPa.

Young modulus is typically measured using the Atomic Force Spectroscopy (AFM) technique. In a typical experiment, the biocompatible nanoparticle is attached or immobilized on a plane surface such as a borosilicate plate. The biocompatible nanoparticle fixed to the surface is immersed in an appropriate medium, typically in PBS or HEPES buffer. The force measurements are typically performed using the “colloidal probe method” [Attachment of micro- and nano particles on tipless cantilevers for colloidal probe microscopy. J. Colloid and Interface Science. 426 (2014) 190-198], where a colloidal particle, typically a silica microsphere, is attached to an atomic force microscope (AFM) cantilever. In the context of the present invention, the spring constant of the cantilever is adapted to the elasticity of the nanoparticle and is typically below 500 mN.m⁻¹. The localization of the nanoparticle on the surface is typically performed by scanning the surface. Approach and retraction force curve is recorded using a constant speed of the tip, typically below 100 nm.s⁻¹. Young modulus is extracted from the experimental curve by fitting the force profile with the appropriate model. The “Herzt model” is typically a well-established model for the calculation [Determining the elastic modulus of biological samples using atomic force microscopy. JPK Instruments Application Note].

A “modified hertz model” can also be used to calculate the Young modulus of the biocompatible nanoparticle. In article “Young's moduli of surface-bound liposomes by atomic force microscopy force measurements” (Langmuir 2008, 24; 2009-2014), authors report Young modulus of liposomes typically below 40 kPa, preferably below 10 kPa. For comparison, a living cell has typically Young modulus values below 100 kPa. The Young modulus of a gel can vary depending on the degree of cross-linking. In their paper “Effect of mechanical properties of hydrogel nanoparticles on macrophages cell uptake” (Soft Matter, 2009, 5; 3984-3991), authors report increase values of Young modulus (from 18.04 kPa up to 211.39 kPa), with crosslinking increase (mol %) in their biocompatible nanoparticles made of N,N-diethyl acrylamide and 2-hydroxyethyl methacrylate crosslinked with N,N′-methylene-bis-acrylamide.

The combined properties, size, surface charge, and the elasticity of the nanoparticles, allow for a short blood circulation of the nanoparticles and extravasation into the liver organ. Moreover, mechanical processes have emerged as important regulators in the complex interplay between cells and their microenvironment. The interaction of cells with the surrounding extra cellular matrix (ECM) is essential for their proper function, as well as the maintenance of tissue architecture and homeostasis. Cells have developed a large repertoire of receptors capable of binding to the ECM. They provide a physical link to the ECM and allow them to transduce signals emanating from the ECM by adapting their behavior to properties of this complex environment. Noteworthy, cell behavior is affected by the composition, the topography and the mechanical properties of the ECM. Cells have typically an elastic modulus (i.e. a young modulus) between 100 Pa and 100 kPa [Demichelis A. et al. Study of AFM Force Spectroscopy method for elastic modulus measurement of living cells. Journal of Physics: Conference Series 459 (2013) 012050]. In the context of the invention, the biocompatible nanoparticles have typically an elasticity (i.e. an elastic modulus or a young modulus) no greater than that of cells for a safe interactions with cells (i.e. without affecting the cell's functions). Therefore, by sequentially administering the biocompatible nanoparticles of the invention and the compound(s) of interest, no co-circulation or a limited co-circulation of the two compounds (i.e. of the biocompatible nanoparticle and of the compound of interest), is achieved. Therefore, the combined properties of the biocompatible nanoparticles, size, surface charge and elasticity, permit the safe use of the compound(s) of interest while allowing (maintaining) the same pharmaceutical (i.e. therapeutic, prophylactic or diagnostic) benefit of the compound(s) for a reduced toxicity thereof for the subject, or in other words while increasing the pharmaceutical benefit of the compound(s) for an equivalent or reduced toxicity thereof for the subject (preferably a reduced toxicity), when compared to pharmaceutical benefit and toxicity induced by the standard pharmaceutical dose of said compound(s), typically in the absence of any nanoparticle.

The nanoparticle can typically be an organic nanoparticle, such as a lipid-based nanoparticle (glycerolipid, phospholipid, sterol lipid, etc.), for instance a solid lipid nanoparticles, a protein-based nanoparticle also herein identified as “protein-nanoparticle” (albumin for instance), a polymer-based nanoparticle (“polymeric nanoparticle”), a co-polymer-based nanoparticle (“co-polymeric nanoparticle”), a carbon-based nanoparticle, a virus-like nanoparticle (for example a viral vector).

The organic nanoparticle may further be a nanosphere (plain nanoparticle) or a nanocapsule (hollow nanoparticle) such as a liposome, a gel, a hydrogel, a micelle, a dendrimer, etc. A mixture of the herein described organic nanoparticles can also be used. The polymer or co-polymer can be of natural or synthetic origin.

Examples of synthetic (artificial) and natural polymers or co-polymers usable in the context of the invention to prepare organic nanoparticles can be selected from polylactic acid (PLA), Poly (lactide-co-glycolic) acid (PLGA), Polyethyleneglycol (PEG), Polyglactin, Polylactide, Polyoxyethylene fatty acid esters, Polypropylene glycol, Polysorbate, Polyvinyl alcohol, Polyacrylamide, Polymethylmethacrylate, Polyalkylcyanoacrylate, Polylactate-co-glycolate,Poly(amido amine), Poly(ethyleneimine), alginate, cellulose and cellulose derivatives polymers, collagen, hyaluronic acid, polyglutamic acid (PGA), actin, polysaccharide, and gelatin.

The nanoparticles used in the herein described compositions are to be biocompatible, i.e. compatible with living tissues. When required by their composition, the nanoparticles are thus to be coated with a biocompatible material to become usable. In a particular embodiment of the invention, the herein mentioned nanoparticle is thus covered with a biocompatible coating.

The biocompatible material can be an agent allowing interaction with a biological target. Such an agent will typically bring a positive or a negative charge on the nanoparticle's surface when the absolute charge of the nanoparticle is of at least 10 mV.

An agent forming a positive charge on the nanoparticle's surface can be for example selected from aminopropyltriethoxisilane or polylysine. An agent forming a negative charge on the nanoparticle surface can be for example selected from a phosphate (for example a polyphosphate, a metaphosphate, a pyrophosphate, etc.), a carboxylate (for example citrate or dicarboxylic acid, in particular succinic acid) or a sulphate.

In a particular embodiment, as long as the absolute charge of the nanoparticle is of at least 10 mV (|10 mV|), the nanoparticle can be coated with a biocompatible material selected from an agent displaying a steric group. Such a group may be selected for example from polyethylene glycol (PEG); polyethylenoxide; polyvinylalcohol; polyacrylate; polyacrylamide (poly(N-isopropylacrylamide)); polycarbamide; a biopolymer; a polysaccharide such as dextran, xylan and cellulose; collagen; a switterionic compound such as polysulfobetain; etc.

The biocompatible coating may advantageously be a “full coating” (complete monolayer). This implies the presence of a very high density of biocompatible molecules creating an appropriate charge on the all surface of the nanoparticle.

The biocompatible coating may further comprise a labelling agent, typically an agent allowing the visualisation of a color using standard imaging equipment.

The combined administration of the at least one biocompatible nanoparticle together with the at least one compound of interest maintains the pharmaceutical (i.e. therapeutic, prophylactic or diagnostic), typically therapeutic, benefit of the compound(s) of interest for a reduced toxicity, or increases the pharmaceutical benefit of the compound(s) for an equivalent or reduced toxicity, for the subject, typically when administered in the subject in need of the compound(s) of interest, between more than 5 minutes and about 72 hours one from each other, when compared to pharmaceutical benefit and toxicity induced by the standard pharmaceutical, typically therapeutic, dose of said compound(s), typically in the absence of any nanoparticle.

In a particular embodiment, the combined administration of the at least one biocompatible nanoparticle and of the at least one compound of interest allows a reduction of at least 10%, preferably at least 15%, of the administered compound(s) therapeutic dose(s), typically when administered in the subject in need of the compound(s) of interest, between more than 5 minutes and about 72 hours one from each other, when compared to the standard therapeutic dose of said compound(s) while maintaining the same therapeutic benefit for an equivalent toxicity or a reduced toxicity (preferably a reduced toxicity) of the compound(s) for the subject; or while increasing the therapeutic benefit for an equivalent or reduced toxicity of the compound(s) for the subject, typically in the absence of any nanoparticle.

In a particular embodiment, nanoparticle(s) are administered with several compounds of interest, typically at least two compounds of interest.

The nanoparticle is preferably cleared from the subject to whom it has been administered typically within 1 hour and 6 weeks, for example 1 month (4 weeks), within 1 hour and 1 month, for example between 1 hour and 3 weeks, or between 1 hour and 2 weeks, or between 1 hour and 1 week, following its administration to a subject in need of the at least one compound of interest.

The material constituting the nanoparticle (including its biocompatible coating when present) is important in determining the biopersistence (i.e. the persistence in the subject's body) of the nanoparticle. The nanoparticle may be regarded as biodegradable when constituted for example of a biodegradable polymer such as PLGA or PLA. Biodegradability facilitates rapid nanoparticle(s) clearance from the subject.

Different molecules or agents can be used according to the present teaching as the at least one compound of interest, typically as the at least one pharmaceutical compound of interest, administered in combination with the at least one biocompatible nanoparticle as described hereinabove. This compound may be a therapeutic, a prophylactic or a diagnostic compound as previously explained. It can be an organic compound or an inorganic compound.

Examples of organic compound usable as the compound of interest can be selected from a biological compound, an antibody, an oligonucleotide, a synthesized peptide, a small molecule targeted therapeutic, an oncolytic virus, a cytotoxic compound, and any corresponding prodrug or derivative thereof, etc.

In a particular embodiment, the compound of interest used in the context of the present invention is an organic compound preferably selected from a biological compound, a small molecule targeted therapeutic, an oncolytic virus, and a cytotoxic compound. In another particular embodiment, the compound of interest is selected from an antibody, an oligonucleotide, and a synthesized peptide.

A biological compound is for instance an antibody, an antibody drug conjugate, preferably a monoclonal antibody (“mAb”), such as infliximab, adalimumab, bevacizumab, rituximab, trastuzumab, ranibizumab, cetuximab, panatimumab; a protein or a recombinant protein such as enbrel (etanercept) or interferon beta-la; a peptide or a recombinant peptide such as insulin glargine or betaseron; a vaccine such as prevnar 13 or gardasil; a biosimilar such as epogin; an enzyme or a recombinant enzyme such as replagal or creon; etc.

An oligonucleotide is for instance an antisense oligonucleotide, an aptamer, such as mipomersen sodium or pursennid, etc.

A synthesized or artificial peptide such as glatiramer acetate or leuprolide acetate.

An oncolytic virus is a therapeutically useful virus that selectively infects and damages cancerous tissues without causing harm to normal tissues. Oncolytic virus is for instance selected from an adenovirus such as Onyx-015, a coxsackie virus such as Catavak, a herpes simplex virus such as talimogene laherparepvec, a maesla virus such as MV-CEA, a newcastle disease virus, a parvovirus, a poliovirus, a reovirus, a Seneca valley virus, a retrovirus, a vaccinia, a vesicular stomatitis virus.

A small molecule targeted therapeutic generally inhibits enzymatic domains on mutated, overexpressed, or otherwise critical protein (potential target in the context of cancer treatment) within the malignant cells. Some therapeutics agents include those that target cell division (for example a aurora-kinase inhibitor or a cyclin-dependent-kinase inhibitor), as well as other biological mechanisms such as protein turnover and chromatin modification (for example a histone-deacetylase inhibitor). Small molecules targeted therapeutics are for instance imatinib, rapamycin, gefitinib, erlotinib, sorafenib, sunitinib, nilotinib, dasatinib, lapatinib, bortezomib, atorvastatin, etc.

A cytotoxic compound is for instance a DNA-modifying agent, such as an anthracycline (for example doxorubicine, daunorubicine, etc.), an alkylating agent (for example melphalan or temozolomide), as well as a drug interfering very precisely with defined physiological mechanisms such as microtubule polymerization (for example taxol), or metabolite synthesis (for example methotrexate). An activable cytotoxic compound is typically used in the context of Photodynamic Therapy (for example photofrin), and is to be activated by an external source such as a laser source to produce its therapeutic effect.

Other typical cytotoxic compounds are typically selected from chemotherapeutic agent as herein described or as known by the skilled oncologist.

A prodrug (for instance capecitabine or irinotecan) is metabolized in its active form in vivo to produce its expected therapeutic effect.

Examples of inorganic compound usable as the at least one compound of interest can be selected from a transition metal coordination complex, a radiopharmaceutical compound, a nanoparticle, etc.

When the compound of interest is a nanoparticle, said nanoparticle is distinct from the “at least one biocompatible nanoparticle” referred to in the pharmaceutical composition as claimed.

Transition metal coordination complexes offer potential advantages over the more common organic-based drugs, including a wide range of coordination numbers and geometries, accessible redox states, ‘tune-ability’ of the thermodynamics and kinetics of ligand substitution, as well as a wide structural diversity. Metal-based substances interact with cell molecular targets, affecting biochemical functions resulting in malignant cell destruction. Transition metal coordination complexes are typically cytotoxic agents (for instance, platinum coordination complexes: cisplatin, carboplatin, oxaloplatin, or ruthenium or gold coordination complexes) acting on DNA structures.

Radiopharmaceutical compounds emit radiations for diagnosis purposes or in order to selectively destroy malignant cells. Typical radiopharmaceuticals may contain for example strontium-89, thallium-201, techtenium-99, samarium-83, etc.

Nanoparticle may be selected typically from a metal oxide nanoparticle (see WO 2009/147214 and WO 2007/118884 for example), a metallic nanoparticle (gold, platinum or silver nanoparticle for instance), a metal sulfide nanoparticle (Bi2S3 for instance), and any mixture thereof (for example a gold nanoparticle covered with hafnium oxide material). The nanoparticle is for example a nanoparticle which can be activated via an external source such as electromagnetic radiation source, ultrasound source, or magnetic source, etc.

The compound of interest, which is administered in combination with a biocompatible nanoparticle as described hereinabove (typically sequentially administered as herein described), may be encapsulated in a carrier or grafted (or bound) to such a carrier according to means known by the skilled person. A typical carrier is for example a liposome (such as DOXIL or ThermoDox which uses thermosensitive lipid), micelle, polymeric (or “polymer”) carrier, hydrogel, gel, co-polymeric carrier, protein carrier, inorganic carrier.

The pharmaceutical composition of the invention (defined by the combination of compound(s) of interest and of nanoparticle(s)) can be used in many fields, particularly in human or veterinary medicine. This composition is typically for use in a subject identified as an animal, preferably a mammal (for example in the context of veterinary medicine), even more preferably a human being, whatever its age or sex.

The pharmaceutical composition of the invention can be used to prevent or treat a disease or disorder selected from a cardiovascular disease, a Central Nervous System (CNS) disease, a gastrointestinal disease, a genetic disorder, a hematological disorder, a hormonal disorder, an immune disorder, an infectious disease, a metabolic disorder, a musculoskeletal disorder, a cancer, a respiratory disease and an intoxication, etc. In a preferred embodiment, the pharmaceutical composition is for use for preventing or treating a disease or disorder selected from a cardiovascular disease, a CNS disease, a cancer, an infectious disease and a metabolic disorder.

In the context of the present invention, the at least one nanoparticle and the pharmaceutical compound(s) are advantageously to be administered in a subject in need of said compound, between more than 5 minutes and about 72 hours one from each other, typically between more than 5 minutes and about 24 hours, preferably between more than 5 minutes or 30 minutes and about 12 hours, in order to optimize the compound(s) pharmaceutical efficacy.

In the present invention, when the at least one nanoparticle and the compound(s) are administered in a subject in need of said compound(s) between more than 5 minutes and about 72 hours one from each other, the absolute surface charge value of the biocompatible nanoparticle is of at least 10 mV (|10 mV|).

In a particular embodiment of the present invention, when the at least one nanoparticle and the compound(s) are administered in a subject in need of said compound(s) between more than 5 minutes and about 24 hours one from each other, the absolute surface charge value of the biocompatible nanoparticle is advantageously of at least 15 mV (|15 mV|).

In another particular embodiment of the present invention, when the at least one nanoparticle and the compound(s) are administered in a subject in need of said compound(s) between more than 5 minutes and about 12 hours one from each other, the absolute surface charge value of the biocompatible nanoparticle is advantageously of at least 20 mV (|20 mV|).

Also herein described is a method of preventing or treating a subject suspected to be predisposed to a disease, or suffering of a disease, such as those herein mentioned, wherein said method comprises administering to said subject a pharmaceutical composition according to the present invention, typically at least one biocompatible nanoparticle and at least one compound of interest as herein described. Anyone of the at least one nanoparticle or of the at least one compound of interest can be administered first to the subject as long as the biocompatible nanoparticle(s) on one side and the compound(s) of interest on the other side are administered separately, typically with an interval of between more than 5 minutes and about 72 hours. Administration of said at least one nanoparticle or at least one compound of interest can be a single administration of each, repeated administrations of each, for example several consecutive administrations of each. The at least one biocompatible nanoparticle may be administered once and the at least one compound of interest may be administered more than once and vice versa.

In a particular embodiment, the biocompatible nanoparticle is at least administered at the beginning of a protocol comprising several administrations of the at least one compound interest, i.e. at least at the first administration of said at least one compound of interest and before or after the administration thereof.

In another particular embodiment, the at least one biocompatible nanoparticle is not administered at the beginning of a protocol comprising several administrations of compound(s) interest and is not administered before the second or third administration of said compound(s) of interest, and before or after the administration thereof.

In the context of these last two embodiments, the biocompatible nanoparticle(s) can also be administered together (before or after as previously explained) with the compound(s) of interest during part or all of the subsequent administrations of said compound(s) of interest.

In a particular embodiment, the at least one nanoparticle of the invention is administered to the subject before administration to said subject of the at least one compound of interest, typically between more than 5 minutes and about 72 hours before administration of the at least one compound of interest.

In this context, the term “nanoparticle” can more particularly refer to a product, in particular a synthetic product, with a size between about 4 nm and about 100 nm, for example between about 10 nm, 15 nm or 20 nm and about 100 nm. An example of compound interest to be used with such a nanoparticle is an organic compound, typically a biological compound. It is advantageously selected from an antibody, an oligonucleotide, a synthesized peptide, a small molecule targeted therapeutic, an oncolytic virus, and a cytotoxic compound and is preferably an antibody, a small molecule targeted therapeutic and/or a cytotoxic compound. The term “nanoparticle” can otherwise refer to a product, in particular a synthetic product, with a size between about 100 nm and about 500 nm, typically between about 100 nm and about 300 nm. An example of compound interest to be used with such a nanoparticle is an inorganic compound, typically selected from a metallic nanoparticle, a metal oxide nanoparticle, a metal sulfide nanoparticle and any mixture thereof or any compound of interest encapsulated in a carrier or grafted to such a carrier.

The biocompatible nanoparticle(s) of the pharmaceutical composition of the invention can be administered by different routes such as subcutaneous, intra venous (IV), intra-dermic, intra-arterial, airways (inhalation), intra peritoneal, intra muscular and/or oral route (per os), preferably by intra venous (IV), intra-arterial, and/or intra peritoneal route.

The compound(s) of interest of the pharmaceutical composition of the invention can be administered by different routes such as subcutaneous, intra venous (IV), intra-dermic, intra-arterial, airways (inhalation), intra peritoneal, intra muscular and/or oral route (per os).

The following examples illustrate the invention without limiting its scope.

### BRIEF DESCRIPTION OF THE FIGURES

FIG. 1: Schematic view of possible routes for therapeutic compounds removal from blood circulation depending on the compound's size (longest dimension).

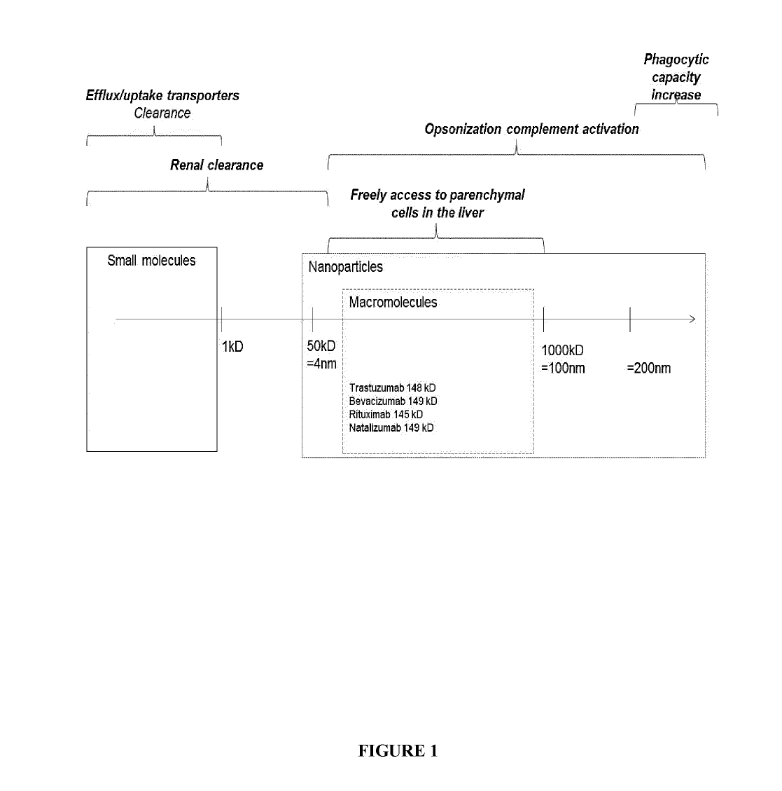

FIG. 2: Schematic representation of the treatments' schedule for the pharmaceutical composition comprising (i) the biocompatible nanoparticles of example 3 and (ii) the Dox-NP® in MDA-MB-231-lucD3H2LN xenografts.

FIG. 3: Tumor re-growth delay of the pharmaceutical composition comprising the biocompatible nanoparticles of example 3 and the Dox-NP® in MDA-MB-231-lucD3H2LN xenografts (mean RTV±SD).

FIG. 4: Chemical formula of L-Glutamic acid, N-(3-carboxy-1-oxopropyl)-,1,5-dihexadecyl ester (SA-lipid)

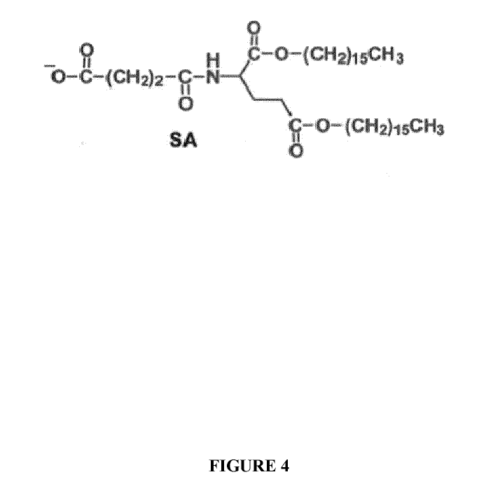

### EXAMPLES

### Example 1

### Synthesis n°1 of Liposomes as Biocompatible Nanoparticles

Liposomes are prepared using the lipidic film re-hydration method:

a) Lipids are solubilized in chloroform. Chloroform is finally evaporated under a nitrogen flow. Re-hydration of the lipidic film with HEPES 20 mM and NaCl 140 mM at pH 7.4 is performed at 50° C., so that the lipidic concentration is 5 mM.

The following lipidic composition was used to prepare charged liposomes: DPPC (DiPalmitoylPhosphatidylCholine): 86% mol; MPPC (MonoPalmitoylPhosphatidylcholine): 10% mol; DSPE-PEG (DiStearylPhosphatidylEthanolamine-[methoxy(PolyElthyleneGlycol)-2000]): 4% mol.

b) Freeze-thaws cycles are then performed 6 times, by successively plunging the sample into liquid nitrogen and into a water bath regulated at 50° C.

c) A thermobarrel extruder (LIPEX™ Extruder, Northern Lipids) was used to calibrate the size of the liposomes under controlled temperature and pressure. In all cases, extrusion was performed at 50° C., under a pressure of 10 bars.

Size distribution of the as-prepared liposomes was determined by dynamic light scattering (DLS) using a Zetasizer NanoZS (Malvern instrument) with a 633 nm HeNe laser at an angle of 90° C. The liposomes suspension was diluted 100 times in HEPES 20 mM and NaCl 140 mM at pH 7.4. Liposome size (i.e. hydrodynamic diameter) was equal to about 170 nm (distribution by intensity) with a polydispersity index (PDI) equal to about 0.1.

As understandable by the skilled person, the desired surface charge was obtained thanks to the selected lipidic composition, and its value was confirmed by zeta potential measurement using a Zetasizer NanoZS (Malvern instrument).

The liposomes were diluted 100 times in water and the pH of the resulting suspension was adjusted to pH 7.4. The liposome surface charge was equal to about −14 mV at pH 7.4.

### Example 2

### Method Allowing a Reduction of at Least 10% of the Dose of Therapeutic Compound to Be Administered in a Subject for an Equivalent therapeutic Efficacy Thereof in the Subject

A pharmaceutical composition according to claim 1 comprising a biocompatible nanoparticle and an activable oxide nanoparticle for anti-cancer therapy (used as “the compound” or “pharmaceutical compound”) which can generate electron and/or high energy photon when exposed to ionizing radiations such as X-rays, is administered in nude mice bearing a xenografted tumor in the following manner:

a) administering to each nude mice (by intra venous injection) the biocompatible nanoparticles;

b) between more than 5 minutes and 72 hours following step a), administering (by intra venous injection) the therapeutic compound in each mice of step a) at a lower dose (10%) when compared to the dose currently used;

c) measuring the therapeutic compound concentration in blood or plasma samples of each mice to obtain the pharmacokinetic parameters of the therapeutic compound, said concentration being measured once or preferably several times between 1 minute and 24 hours following the therapeutic compound administration;

d) assessing any clinical sign of toxicity after the administration of the pharmaceutical composition; and

e) measuring the tumor accumulation of the therapeutic compound 24 hours after its intravenous (IV) administration.

### Example 3

### Synthesis n°2 of Liposomes as Biocompatible Nanoparticles

Liposomes are prepared using the lipid film re-hydration method:

a) Lipids are solubilized in chloroform. Chloroform is finally evaporated under a nitrogen flow. Re-hydration of the lipid film with HEPES 20 mM and NaCl 140 mM at pH 7.4 is performed at 60° C., so that the lipid concentration is 25 mM.

The following lipid composition was used to prepare charged liposomes: DPPC (DiPalmitoylPhosphatidylCholine) 62% mol; HSPC (Hydrogenated Soybean PhosphatidylCholine) 20% mol; CHOL (Cholesterol) 16% mol; POPS (1-Palmitoyl-2-Oleoyl PhosphatidylSerine) 1% mol; DSPE-PEG (DiStearylPhosphatidylEthanolamine-[methoxy(PolyElthyleneGlycol)-2000]) 1% mol.

b) Freeze-thaw cycles are then performed 6 times, by successively plunging the sample into liquid nitrogen and into a water bath regulated at 60° C.

c) a thermobarrel extruder (LIPEX™ Extruder, Northern Lipids) was used to calibrate the size of the liposomes under controlled temperature and pressure. First, 5 passages through a polyethersulfone (PES) 0.45 μm pores-sized membrane were performed at a pressure of 5 bars, then 12 passages through a PES 0.22 μm pores-sized membrane at 10 bars, and finally 12 passages through a 0.1 μm polyvinylidene fluoride (PVDF) membrane at 15 bars. Size distribution of the as-prepared liposomes was determined by dynamic light scattering (DLS) using a Zetasizer NanoZS (Malvern instrument) with a 633 nm HeNe laser at an angle of 90° C. The liposomes suspension was diluted 100 times in HEPES 20 mM and NaCl 140 mM at pH 7.4. Liposome size (i.e. hydrodynamic diameter) was equal to about 145 nm (distribution by intensity) with a polydispersity index (PDI) equal to about 0.1. As understandable by the skilled person, the desired surface charge was obtained thanks to the selected lipidic composition, and its value was confirmed by zeta potential measurement using a Zetasizer NanoZS (Malvern instrument).

The liposomes were diluted 100 times in a sodium chloride solution at 1mM and the pH of the resulting suspension was adjusted to pH 7.4. The liposomes surface charge was equal to about −25 mV at pH 7.4, NaCl 1 mM.

### Example 4

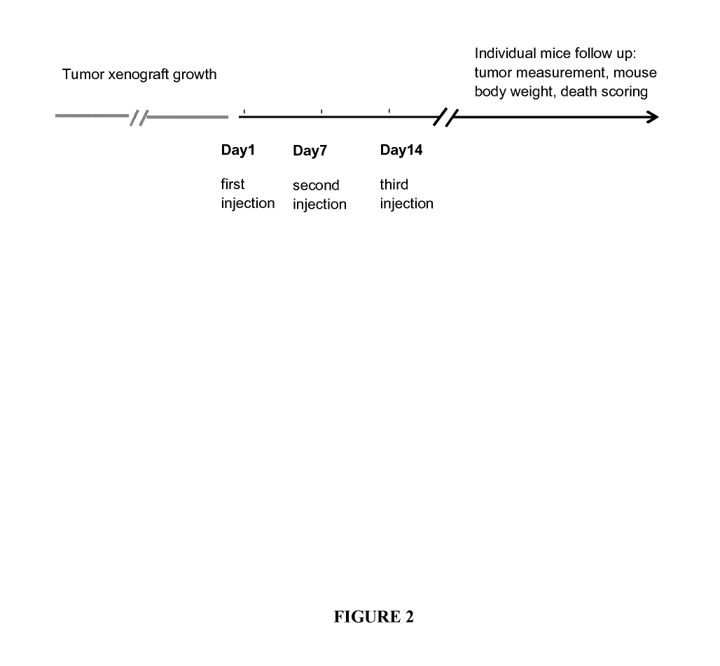

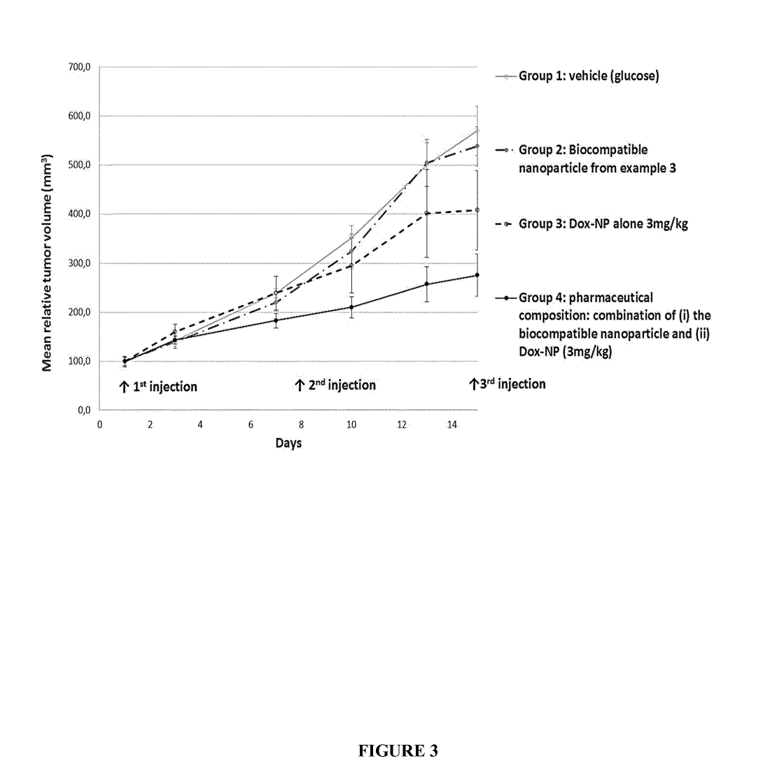

### Tumor Re-Growth Delay of the Pharmaceutical Composition Comprising the Biocompatible Nanoparticles Suspension of Example 3 and the Dox-NPO in MDA-MB-231-lucD3H2LN Xenografts (FIGS. 2 and 3)

This study was performed to investigate the efficacy of the pharmaceutical composition comprising (i) the biocompatible nanoparticle from example 3 and (ii) Dox-NP® (Liposomal Encapsulated Doxorubicin) as the therapeutic compound of interest, in MDA-MB-231-luc-D3H2LN tumor model xenografted on NMRI nude mice.

The human breast adenocarcinoma MDA-MB-231-luc-D3H2LN cell line was purchased at Caliper Life Science (Villepinte, France). The cells were cultured in Minimum Essential Medium with Earl's Balanced Salts Solution MEM/EBSS medium supplemented with 10% fetal bovine serum, 1% non-essential amino acids, 1% L-glutamine, and 1% sodium pyruvate (Gibco).

NMRI nude mice, 6-7 weeks (20-25 g) were ordered from Janvier Labs (France). Mice were subjected to a total body irradiation of 3Gy with the Cesium-137 irradiation device one day before the inoculation of the cancer cells for xenograft.

MDA-MB-231-luc-D3H2LN tumors were obtained by subcutaneous injection of 4.10⁶ cells in 50 μL in the lower right flank of the mouse. The tumor were grown until reaching a volume around about 100 mm³. Tumor diameter was measured using a digital caliper and the tumor volume in mm³ was calculated using the formula:

$$Tumor volume ( {mm}^{3} ) = \frac{length ( mm ) \times {( width )}^{2} ( {mm}^{2} )}{2}$$

Mice were randomized into separated cages and identified by a number (pawn tattoo). Four groups were treated as illustrated on FIG. 2.
Group 1: sterile glucose 5% (control (vehicle) group)

Four (4) mice were intravenously (IV) injected with a sterile glucose 5% solution on day 1, day 7 and day 14. Each time (day), two injections of glucose 5% were performed. The first injection of glucose 5% solution was performed 4 hours before the second injection.
Group 2: Biocompatible nanoparticles from example 3 (control group)

Four (4) mice were intravenously (IV) injected with a sterile glucose 5% solution and the biocompatible nanoparticles from example 3 (10 ml/kg) on day 1, day 7 and day 14. Each time (day), the injection of biocompatible nanoparticles from example 3 was performed 4 hours before injection of the glucose 5% solution.
Group 3: Dox-NP® (3 mg/kg doxorubicin) (treatment group)

Five (5) mice were intravenously (IV) injected with a sterile glucose 5% solution and Dox-NPC® (3 mg/kg doxorubicin) on day 1, day 7 and day 14. Each time (day), the injection of sterile glucose 5% solution was performed 4 hours before the injection of Dox-NP® (3 mg/kg doxorubicin).
Group 4: pharmaceutical composition, i.e. the combination of (i) the biocompatible nanoparticles from example 3 and of (ii) Dox-NP® (3 mg/kg doxorubicin) (treatment group)

Five (5) mice were intravenously (IV) injected with the biocompatible nanoparticles from example 3 (10 ml/kg) and with the Dox-NP® (3 mg/kg doxorubicin) on day 1, day 7 and day 14. Each time (day), the injection of biocompatible nanoparticles from example 3 was performed 4 hours before the injection of Dox-NP® (3 mg/kg doxorubicin).

The Dox-NP® (Avanti Polar lipids—Liposomal formulation of 2 mg/ml doxorubicin HCl at pH 6.5-6.8, in 10 mM histidine buffer, with 10% w/v sucrose) was injected without additional dilution at a volume required to obtain 3 mg/kg of injected doxorubicin.

The biocompatible nanoparticles suspension from example 3 was used without any additional dilution.

The Dox-NP® and the biocompatible nanoparticles from example 3 were administrated by intravenous injection (IV) via lateral tail vein with a 100U (0.3 ml) insulin syringe (TERUMO, France).

Mice were followed up for clinical signs, body weight and tumor size.

The tumor volume was estimated from two dimensional tumor volume measurements with a digital caliper using the following formula:

$$Tumor volume ( {mm}^{3} ) = \frac{length ( mm ) \times {( width )}^{2} ( {mm}^{2} )}{2}$$

In each group, the relative tumor volume (RTV) was expressed as Vt/Vo ratio (Vt being the tumor volume on a given day during the treatment and Vo being the tumor volume at the beginning of the treatment).

The treatment efficacy was determined using the specific growth delay (SGD) over two doubling time (one doubling time being the amount of time it takes for the tumor to double in volume) and the optimal percent T/C value (% T/C).

The SGD was calculated over two doubling time as follows:

$$SGD = \frac{T 4 d treated - T 4 d control}{T 4 d control}$$

with T4d being the time required for the tumor to double twice in volume (mean RTV from 100 mm³ up to 400 mm³).

The Percent T/C value (“% T/C”) was calculated by dividing the median of the relative tumor volume of treated groups (groups 2, 3, 4) versus control group (group 1) at days 1, 3, 7, 10, 13, 15, 18, 21 and 24, and by multiplying the result of said division by 100 (see Table 2). The lowest % T/C values obtained within 2 weeks following treatment injection (with or without biocompatible nanoparticles as used in the context of the present invention) correspond to the optimal % T/C values.

FIG. 3 shows the mean relative tumor volume (mean RTV) for all groups as obtained (in the conditions previously described) after IV injections of:
vehicle (sterile glucose 5%) on day 1, 7 and 14 (group 1);
biocompatible nanoparticles from example 3, 4 hours prior the vehicle (sterile glucose 5%) injection on day 1, 7 and 14 (group 2);
Dox-NP® (3 mg/kg doxorubicin) on day 1, 7 and 14 (group 3); or
biocompatible nanoparticles from example 3, 4 hours prior the Dox-NP® (3 mg/kg doxorubicin) injection on day 1, 7 and 14 (group 4).

As shown on FIG. 3, a marked tumor growth inhibition is observed after the first injection of the pharmaceutical composition comprising the combination of (i) the biocompatible nanoparticles from example 3 and (ii) the Dox-NP® (3 mg/kg doxorubicin), when compared to the Dox-NP® (3 mg/kg doxorubicin) alone.

The time required (expressed in days) for each tumor to double twice in volume (T4d) was calculated (as a measure of the duration of the treatment effects). T4d for the pharmaceutical composition was estimated to about 31 days versus about 14 days for the Dox-NP® alone (table 1). In addition the Specific Growth Delay (SGD) estimated from the tumors growth over two doubling time (starting from a mean RTV of 100 mm³ up to 400 mm³) was equal to about 2 for the pharmaceutical composition versus about 0 for the Dox-NP® alone (table 1).

TABLE 1
Time for the tumor to double twice in volume (T4d) and Specific
Growth Delay (SGD) estimated from the tumors growth over two
doubling time. Td4 represents the number of days to reach two
doubling time (mean RTV from 100 mm³ up to 400 mm³).
The control group is the vehicle (glucose 5%) alone (—).

| | T4d (in days) between 100 | |
|---|---|---|
| Groups | and 400 mm³ (mean RTV) | SGD |
| Group 1: vehicle (control group) | 11 | — |
| Group 2: Biocompatible nanoparticles | 11 | 0 |
| from example 3 | | |
| Group 3: Dox-NP ® alone (3 mg/Kg) | 14 | 0 |
| Group 4: Pharmaceutical composition | 31 | 2 |
| comprising (i) the biocompatible | | |
| nanoparticle from example 3 and (ii) | | |
| Dox-NP ® (3 mg/Kg) | | |

Furthermore, the percent T/C (% T/C) (calculated until the day of sacrifice of group 1) decrease faster for the pharmaceutical composition than for Dox-NP® alone. This demonstrates a marked impact of the pharmaceutical composition. The optimal % T/C of 25 observed at day 24 was indeed obtained for the pharmaceutical composition, i.e. the combination of (i) the biocompatible nanoparticles from example 3 and (ii) the Dox-NP® (3 mg/kg doxorubicin), whereas the optimal % T/C of 38 observed at day 21 was obtained for the group Dox-NP® alone (Table 2).

Table 2: percent T/C (% T/C) is calculated by dividing the median of the relative tumor volume of treated groups (groups 2, 3, 4) versus control group (group 1) at days 1, 3, 7, 10, 13, 15, 18, 21 and 24, and by multiplying the result of said division by 100. Control group is group 1 (vehicle sterile glucose 5% alone). % T/C is calculated until day 24 which correspond to the day of sacrifice of group 1 (control group). Optimal % T/C is indicated for each group in gray boxes.

Overall, those result showed an advantageous tumor growth delay when using the pharmaceutical composition of the present invention [corresponding to the combination of (i) the biocompatible nanoparticles from example 3 and of (ii) the Dox-NP® (3 mg/kg doxorubicin)], which is not observed when the Dox-NP® (3 mg/kg doxorubicin) is used alone (i.e. in the absence of the biocompatible nanoparticles used in the context of the present invention). This tumor growth delay was observed when the biocompatible nanoparticles from example 3 and the compound of interest (the Dox-NPC) were administered sequentially, the biocompatible nanoparticle being administered to the

subjects 4 hours before the Dox-NP®.

### Example 5

### Synthesis n°3 of Liposomes as Biocompatible Nanoparticles

Liposomes are prepared using the lipid film re-hydration method:

a) Lipids are solubilized in chloroform. Chloroform is finally evaporated under a nitrogen flow to form a lipid film on the Pyrex tube walls. Re-hydration of the lipid film with HEPES 25 mM and NaCl 150 mM at pH 7.4 is performed at 60° C., so that the lipid concentration is 50 mM.

The following lipid composition was used to prepare charged liposomes: DPPC (DiPalmitoylPhosphatidylCholine) 58% mol; HSPC (Hydrogenated Soybean PhosphatidylCholine) 21% mol; CHOL (Cholesterol) 16% mol; POPS (1 -Palmitoyl-2-Oleoyl PhosphatidylSerine) 5% mol.

b) Freeze-thaw cycles are then performed 6 times, by successively plunging the sample into liquid nitrogen and into a water bath regulated at 60° C. Ultra-sonication of the liposomes solution is performed during 30s every 3 freeze-thaw cycles and just before extrusion.

c) A thermobarrel extruder (LIPEX™ Extruder, Northern Lipids) is used to calibrate the size of the liposomes under controlled temperature and pressure. Extrusion is performed at 60° C. Ten passages are applied through a 0.1μm pores size polyvinylidene fluoride (PVDF) membrane under a pressure of 10 bars.

Size distribution of the as-prepared liposomes is determined by dynamic light scattering (DLS) using a Zetasizer NanoZS (Malvern instrument) with a 633 nm HeNe laser at an angle of 173° C. The liposomes solution is diluted 200 times in HEPES 25 mM and NaCl 150 mM at pH 7.4. Liposomes size (i.e. hydrodynamic diameter) is equal to about 170 nm (distribution by intensity) with a polydispersity index (PdI) equal to about 0.2.

As understandable by the skilled person, the desired surface charge is obtained thanks to the selected lipid composition, and its value is confirmed by zeta potential measurement using a Zetasizer NanoZS (Malvern instrument). The liposomes are diluted 200 times in a sodium chloride solution at 1mM and the pH of the solution is adjusted to pH 7. The liposomes surface charge is equal to about −40 mV at pH 7, NaCl 1 mM.

The final lipid concentration of the liposomes solution is measured by a colorimetric assay (Bartlett method). The method is based on total phosphorus determination through an acidic digestion of phospholipid. The released inorganic phosphate is reacted with ammonium molybdate, the complex giving a strong blue color. Lipids concentration is equal to about 50 mM.

### Example 6

### Synthesis n°4 of Liposomes as Biocompatible Nanoparticles

Liposomes are prepared using the lipid film re-hydration method:

a) Lipids are solubilized in chloroform. Chloroform is finally evaporated under a nitrogen flow to form a lipid film on the Pyrex tube walls. Re-hydration of the lipid film with

HEPES 25 mM and NaCl 150 mM at pH 7.4 is performed at 60° C., so that the lipid concentration is 50 mM.

The following lipid composition was used to prepare the charged liposomes: DPPC (DiPalmitoylPhosphatidylCholine) 45.15% mol; CHOL (Cholesterol) 45.15% mol; DSPE-PEG (DiStearylPhosphatidylEthanolamine-[methoxy(PolyElthyleneGlycol)-2000]) 0.60% mol; L-Glutamic acid, N-(3-carboxy-1-oxopropyl)-, 1,5-dihexadecyl ester (SA-lipid) 9.10% mol. The SA-lipid brings COOH groups on the liposomes surface.

b) Freeze-thaw cycles are then performed 6 times, by successively plunging the sample into liquid nitrogen and into a water bath regulated at 60° C.

c) A thermobarrel extruder (LIPEX™ Extruder, Northern Lipids) is used to calibrate the size of the liposomes under controlled temperature and pressure. Extrusion is performed at 60° C. Seven passages are applied through a 0.45μm pores size polyvinylidene fluoride (PVDF) membrane under a pressure of 3 bars and ten passages through a 0.22μm pores size polyvinylidene fluoride (PVDF) membrane under a pressure of 10 bars. Size distribution of the as-prepared liposomes is determined by dynamic light scattering (DLS) using a Zetasizer NanoZS (Malvern instrument) with a 633 nm HeNe laser at an angle of 173° C. The liposomes solution is diluted 200 times in HEPES 25 mM and NaCl 150 mM at pH 7.4. Liposomes size (i.e. hydrodynamic diameter) is equal to about 230 nm (distribution by intensity) with a polydispersity index (PdI) equal to about 0.2.

As understandable by the skilled person, the desired surface charge is obtained thanks to the selected lipid composition, and its value is confirmed by zeta potential measurement using a Zetasizer NanoZS (Malvern instrument). The liposomes solution is diluted 200 times in a sodium chloride solution at 1mM and the pH of the solution is adjusted to pH 7. The liposomes surface charge is equal to about −60 mV at pH 7, NaCl 1 mM.

The final lipid concentration of the liposomes solution is measured by a colorimetric assay

(Bartlett method). The method is based on total phosphorus determination through an acidic digestion of phospholipid. The released inorganic phosphate is reacted with ammonium molybdate and the complex giving a strong blue color. Lipids concentration is equal to about 50 mM.

### Example 7

### Synthesis n°5 of Liposomes as Biocompatible Nanoparticles

Liposomes are prepared using the lipid film re-hydration method:

a) Lipids are solubilized in chloroform. Chloroform is finally evaporated under a nitrogen flow to form a lipid film on the Pyrex tube walls. Re-hydration of the lipid film with HEPES 25 mM and NaCl 150 mM at pH 7.4 is performed at 60° C. and the lipid concentration is 50 mM. The following lipid composition was used to prepare the charge liposomes: DSPC (1,2-distearoyl-sn-glycero-3-phosphocholine) 60% mol, CHOL (Cholesterol) 35% mol; and Succinyl PE (1,2-dioleoyl-sn-glycero-3 -phosphoethanolamine-N-succinyl) 5% mol.

b) Freeze-thaw cycles are then performed 6 times, by successively plunging the sample into liquid nitrogen and into a water bath regulated at 60° C. Ultra-sonication of the liposomes solution is performed during 30 s, every 3 freeze-thaw cycles and just before extrusion.

c) A thermobarrel extruder (LIPEX™ Extruder, Northern Lipids) is used to calibrate the size of the liposomes under controlled temperature and pressure. Extrusion is performed at 60° C. Twelve passages are applied through a 0.22 μm pores size polyvinylidene fluoride (PVDF) membrane under a pressure of 12 bars.

d) Conjugation of p-aminophenyl-α-D-mannopyranoside (MAN) to Succinyl PE liposome: The succinyl PE liposome surface are modified with a mannose derived ligand p-aminophenyl-α-D-mannopyranoside (MAN), using carbodiimide coupling to develop mannose conjugated liposome. MAN is covalently coupled by its amino group to the carboxylic acid group of Succinyl PE, present on the surface of preformed Succinyl PE liposome. Briefly, to the preformed Succinyl PE liposome solution are added EDC (1-ethyl-3-[3-dimethylaminopropyl] carbodiimide hydrochloride), (Succinyl PE/EDC 1:10 molar ratio) and N-hydroxysuccinimide (NHS) (NHS/EDC 1:2.5 molar ratio). The pH of the suspension is then adjusted at 6 with NaOH 1M and the resulting suspension is stirred for 15 minutes at room temperature. Subsequently, the pH of the solution is adjusted at 7 with NaOH 1M and the aqueous MAN solution is added (Succinyl PE/MAN 1:2 molar ratio) to the solution. pH is readjusted at 7 using NaOH 1M and the suspension is stirred for 2 additional hours at room temperature. Excessive unbound MAN, EDC and NHS molecules are removed by 3 steps of dialysis with dilution factor (×500; ×500; ×500) using a 50 KDa cellulose membrane.

Of note, due to possible dilution upon dialysis, the liposomes solution can be concentrated by centrifugation (typically a Sigma 3-15K centrifuge at 5° C.; 1,200 rpm) using membrane ultrafiltration on Vivaspin concentrators with a polyethylene sulfone (PES) membrane and a cut-off 300 KDa.

Size distribution of the as-prepared liposomes is determined by dynamic light scattering (DLS) using a Zetasizer NanoZS (Malvern instrument) with a 633 nm HeNe laser at an angle of 173° C. The liposomes solution is diluted 200 times in HEPES 25 mM and NaCl 150 mM at pH 7.4. Liposomes size (i.e. hydrodynamic diameter) is about 230 nm (distribution by intensity) with a polydispersity index (PDI) around 0.2.

As understandable by the skilled person, the desired surface charge is obtained thanks to the selected lipid composition, and its value is confirmed by zeta potential measurement using a Zetasizer NanoZS (Malvern instrument). The liposomes solution is diluted 200 times in a sodium chloride solution at 1 mM and at pH 7. The liposomes surface charge is around −70 mV at NaCl 1 mM, pH 7.

The final lipid concentration of the liposomes solution is measured by a colorimetric assay (Bartlett method). The method is based on total phosphorus determination through an acidic digestion of phospholipid. The released inorganic phosphate is reacted with ammonium molybdate and the complex giving a strong blue color. Lipids concentration is equal to about 50 mM.

1-13. (canceled)

14. A therapeutic, prophylactic or diagnostic method comprising a step of administering at least one pharmaceutical compound to a subject in need thereof and a distinct step of administering at least one biocompatible nanoparticle, wherein the longest dimension of the biocompatible nanoparticle is between about 4 nm and about 500 nm, the absolute surface charge value of the at least one biocompatible nanoparticle is of at least |110 mV|, the Young modulus of the at least one biocompatible nanoparticle is less than 100 kPa, the at least one biocompatible nanoparticle is not used as a pharmaceutical compound, and said at least one nanoparticle is administered to the subject between 5 minutes and about 72 hours before or after the at least one pharmaceutical compound.

15. The method according to claim 14, wherein the nanoparticle has an absolute surface charge value of more than 10 mV, said charge being a negative charge.

16. The method according to claim 14, wherein the nanoparticle is an organic nanoparticle.

17. The method according to claim 16, wherein the nanoparticle is selected from a lipid-based nanoparticle, a protein-based nanoparticle, a polymer-based nanoparticle, a co-polymer-based nanoparticle, a carbon-based nanoparticle, and a virus-like nanoparticle.

18. The method according to claim 14, wherein the nanoparticle is further covered with a biocompatible coating.

19. The method according to claim 14, wherein the administration of the at least one biocompatible nanoparticle and of the at least one pharmaceutical compound maintains the therapeutic benefit of the pharmaceutical compound and reduces toxicity, or increases the therapeutic benefit of the pharmaceutical compound for an equivalent or reduced toxicity, for the subject, when compared to therapeutic benefit and toxicity induced by the standard therapeutic dose of said pharmaceutical compound in the absence of any nanoparticle.

20. The method according to claim 14, wherein the administration of the at least one biocompatible nanoparticle and of the at least one pharmaceutical compound allows a reduction of at least 10% of the administered pharmaceutical compound therapeutic dose when compared to the standard therapeutic dose of said pharmaceutical compound while maintaining the same therapeutic benefit for an equivalent toxicity or a reduced toxicity for the subject or while increasing the therapeutic benefit for an equivalent or reduced toxicity for the subject.

21. The method according to claim 14, wherein the at least one nanoparticle is cleared from the subject to whom it has been administered within one hour and six weeks after its administration to a subject in need of the pharmaceutical compound of interest.

22. The method according to claim 14, wherein the pharmaceutical compound is an organic compound selected from a biological compound, a small molecule targeted therapeutic, an oncolytic virus, and a cytotoxic compound.

23. The method according to claim 22, wherein the pharmaceutical compound is selected from an antibody, an oligonucleotide, and a synthesized peptide.

24. The method according to claim 14, wherein the pharmaceutical compound is an inorganic compound selected from a metallic nanoparticle, a metal oxide nanoparticle, a metal sulfide nanoparticle and any mixture thereof.

25. The method according to claim 14, wherein the pharmaceutical compound is encapsulated in a carrier.

26. The method according to claim 14, wherein the pharmaceutical compound is bound to a carrier.
